(19) United States
(12) Patent Application Publication
(10) Pub. No.: US 2025/0064619 A1
(43) Pub. Date: Feb. 27, 2025

(54) ANIMAL HOCK SPLINT

(71) Applicant: Iowa State University Research Foundation, Inc., Ames, IA (US)

(72) Inventor: Rodney Steven Bagley, Stanhope, IA (US)

(21) Appl. No.: 18/811,120

(22) Filed: Aug. 21, 2024

Related U.S. Application Data
(60) Provisional application No. 63/578,016, filed on Aug. 22, 2023.

(51) Int. Cl. A61F 5/01; A61D 9/00

(52) U.S. Cl. CPC A61F 5/0127; A61D 9/00; A61F 2005/0172; A61F 2005/0179

(57) ABSTRACT

A movable appendage splint that mimics natural movements can aid an animal, such as a dog, to provide support for natural movement of joints. The splint allows the animal with musculoskeletal or neurologic condition of the pelvic limbs return to normal gaiting activities. A number of rigid portions are connected to one another in an articulating manner and resilient connections, such as springs, allows the articulating movement while urging the portions of the splint back to a previous position. The articulating movement and resilient members will allow the animal to extend and flex joints, while also aiding in returning to the unflexed position to urge normal movement, even if the animal is injured or recovering.

### CROSS REFERENCE TO RELATED APPLICATIONS

This application claims priority under 35 U.S.C. § 119 to provisional patent application U.S. Ser. No. 63/578,016, filed Aug. 22, 2023. The provisional patent application is herein incorporated by reference in its entirety, including without limitation, the specification, claims, and abstract, as well as any figures, tables, appendices, or drawings thereof.

### TECHNICAL FIELD

The present disclosure relates generally to apparatuses, systems, and/or methods of use having application in at least the veterinary industry. More particularly, but not exclusively, the disclosure relates to apparatuses, systems, and/or methods for use in the care and recovery of animals, including dogs, having leg injuries.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Four-legged animals, such as dogs, may have difficulty recovering from injuries and/or treatment to their legs. This may cause issues during the recovery of the patient. For example, the patient may not be able to support the leg, may not be able to place any load or weight on the leg, and/or may not be able to bend portions of the leg. This can cause longer term issues, such as loss of muscle, further damage due to dragging the leg/foot, or other issues to the leg or other extremities.

It is known to provide splints for legs of such animals, which may be used to protect the leg at or below the shoulder joint or hip joint. Such splints generally keep the leg in a straight or other fixed position. However, this is not always desirable, as the patient will not be able to flex joints of the leg. While the leg may be able to heal in a controlled manner, the lack of dynamic movement in the joints can cause unwanted side effects that could cause greater issues for the patient once the animal has otherwise healed. The lack of movement can cause muscle/tendon/ligament contraction, stiffness or make it difficult for the patient to move in a more natural and dynamic manner to promote further recovery and buildup of muscles, which are needed for full recovery.

Thus, there exists a need in the art for an apparatus that improves over existing splints for legs of animals.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of any of the aspects of any of the embodiments of the present disclosure to improve on or overcome the deficiencies in the art.

It is a further object, feature, and/or advantage of any of the aspects of any of the embodiments of the present disclosure to provide support for natural movement of leg joints for animals with injuries. For example, the leg joint could be a carpal joint.

It is still yet a further object, feature, and/or advantage of any of the aspects of any of the embodiments of the present disclosure to aid in mimicking natural movement of joints for animals during recovery of injuries or for animals having leg or joint conditions.

It is another object, feature, and/or advantage of any of the aspects of any of the embodiments of the present disclosure to allow for animals with musculoskeletal or neurologic condition of the pelvic limbs return to normal gaiting activities.

The apparatus/system disclosed herein can be used in a wide variety of applications. For example, while it has been disclosed that the apparatus can be used during the recovery of an animal, the device could also be used to aid in supporting elderly patients, as well as patients of different animal types.

It is preferred that the apparatus be safe, cost effective, and durable. For example, the device can be made to withstand patients (i.e., animals) of various sizes as well as different abilities.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the apparatus.

Methods can be practiced which facilitate use, manufacture, assembly, maintenance, and repair of an apparatus which accomplish some or all of the previously stated objectives.

The apparatus can be incorporated into systems or kits which accomplish some or all of the previously stated objectives.

According to some aspects of the present disclosure, a naturally dynamic/movable splint for animals comprises a first rigid splint member for supporting a first portion of an animal appendage; a second rigid splint member rotatably connected to the first rigid splint member, the second rigid splint member configured to support a second portion of the animal appendage; and a resilient member connecting the first and second rigid splint members, wherein the first and second rigid splint members able to rotate relative to one another upon application of a force and the resilient member urging the first and second rigid splint members to a non-rotated relationship upon easing of the force.

According to at least some aspects of some embodiments, the movable splint further comprises a third rigid splint member rotatably connected to the second rigid splint member.

According to at least some aspects of some embodiments, the movable splint further comprises a second resilient member connecting the second and third rigid splint members.

According to at least some aspects of some embodiments, the third rigid splint member configured to receive a paw portion of the animal appendage.

According to at least some aspects of some embodiments, the resilient member comprises a tension element/spring.

According to at least some aspects of some embodiments, the resilient member comprises a plurality of resilient members each connecting the first and second rigid splint members.

According to at least some aspects of some embodiments, the first and second rigid splint members supporting an ankle of an animal.

According to at least some aspects of some embodiments, the movable splint further comprises padding on the first and second rigid splint members.

According to at least some aspects of some embodiments, the padding on the first and second rigid splint members comprises neoprene.

According to at least some aspects of some embodiments, the movable splint further comprises one or more straps for holding the splint on the animal.

According to additional aspects of the disclosure, an animal splint comprises a plurality of rigid splint members, each of the plurality of rigid splint members conforming to a portion of an animal's appendage; wherein each of the plurality of rigid splint members is rotatably connected to, and articulating with, an adjacent rigid splint member via corresponding axes of the rigid splint members; and at least one resilient member between at least one pair of adjacent rigid splint members, the at least one resilient member urging the at least one pair of adjacent rigid splint members into an initial position.

According to at least some aspects of some embodiments, the plurality of rigid splint members comprises an upper, a mid-level, and a lower rigid splint member, wherein the mid-level rigid splint member is rotatably connected to, and articulating with, the upper rigid splint member at an upper position, and is rotatably connected to the lower rigid splint member at a lower position.

According to at least some aspects of some embodiments, the animal split a first resilient member between the upper and mid-level rigid splint members, and a second resilient member between the mid-level and lower rigid splint members.

According to at least some aspects of some embodiments, the lower rigid splint member includes a substantially planar portion and partially upstanding sidewalls configured to position a paw of an animal.

According to at least some aspects of some embodiments, the at least one resilient member comprises a tension spring.

According to at least some aspects of some embodiments, the animal splint comprises padding in one or more of the plurality of rigid splint members.

According to at least some aspects of some embodiments, the animal splint comprises one or more straps associated with one or more of the plurality of rigid splint members.

According to at least some aspects of some embodiments, the at least one resilient member comprises a rigid plastic.

According to additional aspects, an articulating animal splint comprises a plurality of rigid splint members, each of the plurality of rigid splint members conforming to a portion of an animal's appendage; wherein each of the plurality of rigid splint members is rotatably connected to, and articulating with, an adjacent rigid splint member via corresponding axes of the rigid splint members; at least one resilient member between at least one pair of adjacent rigid splint members, the at least one resilient member urging the at least one pair of adjacent rigid splint members into an initial position; wherein the plurality of rigid splint members comprises an upper, a mid-level, and a lower rigid splint member, wherein the mid-level rigid splint member is rotatably connected to the upper rigid splint member at an upper position, and is rotatably connected to, and articulating with, the lower rigid splint member at a lower position; and padding in one or more of the plurality of rigid splint members.

According to at least some aspects of some embodiments, the at least one resilient member comprises a tension spring; spring steel; rubber; or other elastomers.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a view of a dog dragging its pelvic limbs, such as due to a neurological injury or other malady.

FIG. 2 is view of a naturally dynamic movable splint for animals without padding and showing aspects of the present disclosure.

FIG. 3 is a view of the naturally dynamic movable splint with padding and straps according to aspects of the present disclosure.

FIG. 4 is a view of pelvic limbs of an animal with naturally dynamic movable splints placed on the animal.

FIG. 5 is a view showing the animal with the naturally dynamic movable splints beginning to walk with support from the splints.

FIG. 6 is a view similar to FIG. 5 showing the animal with splints walking using the naturally dynamic movable splints disclosed herein.

FIG. 7 is an enlarged view showing an animal with splints.

FIG. 8 is a view showing additional aspects of a naturally dynamic movable splint for use with animals to aid in supporting walking or other movements of the animal.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

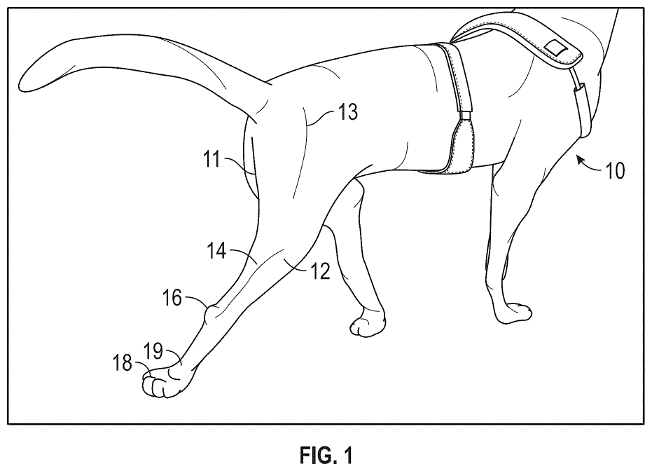

FIG. 1 is a view showing an animal 10, which may be a dog or any other four legged mammal. While the anatomy of such animals may be similar, for purposes of the present disclosure, attention is given to the pelvic limb 11, which is a rear appendage (i.e., leg). It is noted that both pelvic limbs 11 are similar in anatomy, and therefore, while certain portions of the limb will be called out, the same will be applicable for the other limb. In addition, as will be understood, certain parts or portions of the limb will be called out in an effort to educate and/or help understand aspects of novel features of the present disclosure. However, portions of the limb that are not specifically called out herein may be important or otherwise be benefitted by the systems and apparatus of the present disclosure.

Therefore, returning to FIG. 1, the animal's pelvic limb 11 includes an upper thigh 13 and a lower calf 14. A knee 12 is shown, which is a joint to allow bending of the limb 11. Below the knee 12 are bones that extend to the hock or ankle joint 16, which is another joint that allows bending and movement of the limb 11. The canine hock joint 16, also known as the tibiotarsal joint, located on a dog's back leg below the stifle (knee), corresponds to the ankle joint of a human. The hock creates that sharp angle at the back of the dog's rear legs. Below the hock joint 16 are the digits/paw 18, which are connected via the metatarsal joint 19. The metatarsal joint 19 is the joint between the metatarsal bones (the bones that connect the toes to the ankle of the dog) and the phalanges (the three bones in each toe).

As shown in FIG. 1, injuries or conditions, which may be musculoskeletal or neurological, can affect the animal's ability to move or control some of the joints of the limb. Therefore, in an effort to provide support for natural movement of the hock joint 16 of animals with injuries, a movable splint 20 can be used.

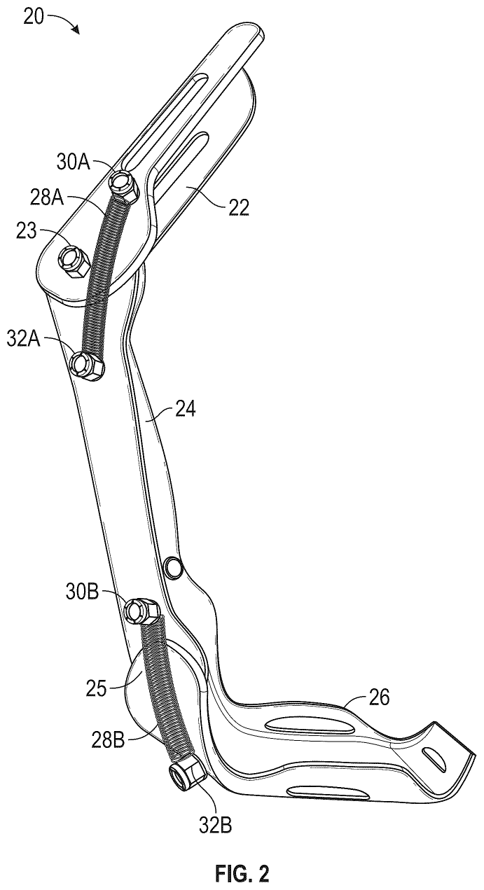

FIG. 2 is a view showing aspects of embodiments of such a naturally dynamic movable dog splint 20 to aid in promoting natural movement of limbs 11 of dogs. The device contains a combination of rigid support and articulative regions to allow animals with musculoskeletal or neurologic condition of the pelvic limbs return to normal gaiting activities. The orthotic device comprises three rigid components, which support the caudal aspect of the tibial and metatarsal regions as well as the plantar aspect of the digits.

For example, as shown in FIG. 2, there is a first rigid support member 22, second rigid support member 24, and third rigid support member 26. The rigid support members can comprise moldable orthopedic plastic, which allows for customization for animals of different size, weight, and other considerations. Orthotists and prosthetists prefer a variety of sheet plastics, including Polypropylene, Kydex, and Copolymer Polypropylene, with Polypropylene being the most extensively used and acceptable. According to at least some aspects of some embodiments, the device 20 would be custom designed and fitted for individual patients. Still further, it should be appreciated that the device 20, or even members of the device, may be predesigned to fit animals. For example, the portions could be ready to fit dogs of certain breeds or sizes, making the device more easily to use when needed.

It is noted that the shape of the members are to generally correspond with anatomical features of the patient animal. For example, the third rigid member 26 includes upwardly raised borders or walls, which are used to hold the paw of the patient in the member. These can be sized and/or custom shaped based on the animal who will be wearing the device. The other rigid members are also shaped to at least partially surround the patient's leg to provide comfort and support for the patient during use. Having the portions be moldable or customizable allows the members to be fit for different sizes of leg portions.

As further noted, the rigid support members are articulated, which means that they are movable relative to one another. This movement can occur due to the members being rotatable relative to one another, such as being rotatably connected to, or articulating with, one another. The rigid members are connected to one another at axes, such as the first axis 23 and the second axis 25. The axes 23, 25 include pins between corresponding holes in the rigid support members that allow such rotational movement. The rotation will allow the otherwise rigid members to act similar to the natural joints of the limb, which will encourage a more natural movement of the animal during use of the device 20.

The pins can generally be any mechanical or non-mechanical fastener that will hold the members together while also allowing the rotational movement of the members. In general, a mechanical fastener is a device that is used to mechanically join or fasten two or more objects together. In general, fasteners are used to create non-permanent joints or connections; that is, joints that can be removed or dismantled without damaging the joining components. General types of mechanical fasteners can include threaded (bolts, screws, nuts, studs, etc.) or non-threaded (keys, pins, retaining rings, etc.). Additional fasteners can include, but are not limited to nails, rivets, and the like. Non-mechanical fasteners may include adhesives, fittings, clearance fittings, friction fittings, compression fittings, transition fittings, snaps, snap fits, hook and loops, joints, and the like. For simplistic purposes, screws, nuts, bolts, pins, rivets, staples, washers, grommets, latches (including pawls), ratchets, clamps, clasps, flanges, ties, adhesives, welds, any other known fastening mechanisms, or any combination thereof may be used to facilitate fastening, may be used for any of the connections described herein and all are to be considered swappable with one another for any of the attachment, connection, and/or fastening of components, either temporarily or permanently. It is further considered that any combination of any of the listed mechanical and/or non-mechanical fasteners or methods of fastening are to be considered a part of the disclosure.

Also shown in the figure are a number of resilient members that are connected between adjacent rigid support members. There is an upper resilient member 28A between the first and second rigid support members and a lower resilient member 28B between the second and third rigid support members. The resilient members 28 are shown to be springs, such as tension springs. However, other resilient and/or elastic members may be used.

Resilient materials are those that can withstand stress, strain, impact, or other external forces without undergoing significant deformation or damage. They have the ability to return to their original shape or state after being subjected to these forces. Some examples of resilient materials that may be used are, but are not limited to:

Rubber: Natural rubber and synthetic rubber are both known for their elasticity and ability to return to their original shape after being stretched or compressed. They are commonly used in tires, shock absorbers, gaskets, and various industrial applications.

Spring Steel: Spring steel is a type of high-carbon steel that can be repeatedly bent, twisted, or compressed without permanent deformation. It is used to make springs for various mechanical applications.

In addition, elastic materials are those that can deform under the application of stress or force but return to their original shape when the stress is removed. These materials exhibit a high degree of elasticity, allowing them to undergo reversible deformation. Some nonlimiting examples of elastic materials are:

Rubber Bands: Rubber bands are a classic example of elastic materials. They can be stretched significantly and will return to their original size and shape when released.

Steel Springs: Steel springs, such as those used in mechanical devices or mattresses, are designed to deform under force and then spring back to their original shape.

Elastomers: Elastomers are polymers that have a high degree of elasticity. Examples include natural rubber and synthetic elastomers like silicone rubber and neoprene.

Bungee Cords: Bungee cords are made from elastic materials and are used for various applications, such as securing objects during transport. They can stretch and then recoil to their original length.

Stretchable Fabrics: Fabrics made from elastic fibers like spandex (Lycra) are used in clothing and sportswear to provide stretchiness and comfort.

Balloon Rubber: The rubber used in balloons is elastic. When you blow air into a balloon, it stretches, and when you release the air, it returns to its original size.

Elastic Bands: Similar to rubber bands, elastic bands are often used to hold items together and can stretch and contract repeatedly.

Elastic Tapes and Straps: Elastic tapes and straps are used in various applications, such as medical bandages, athletic supports, and clothing accessories.

Elastic Hair Ties: Hair ties made from elastic materials are used to tie up hair and can stretch and contract without losing their elasticity.

Elastic Cordage: Elastic cords, ropes, and bungee cords are used in various applications where the ability to stretch and recoil is important.

Again, these are just some examples of materials that can be used, but they are not the only materials and are not to be limiting on the present disclosure. In either sense, the resilient members 28 are used to provide a more natural articulating movement of the rigid components and will promote a healthier recovery via use of the device 20, as will be understood. The resilient members 28 can be connected, such as via mechanical or non-mechanical fasteners, to the rigid members in a rotatable manner. This can be done via an upper attachment axis 30 and a lower attachment axis 32. The resilience of the materials, such as having them be spring loaded, allows for natural motion of the hock and metatarsal/phalangeal joints in both flexion and extension.

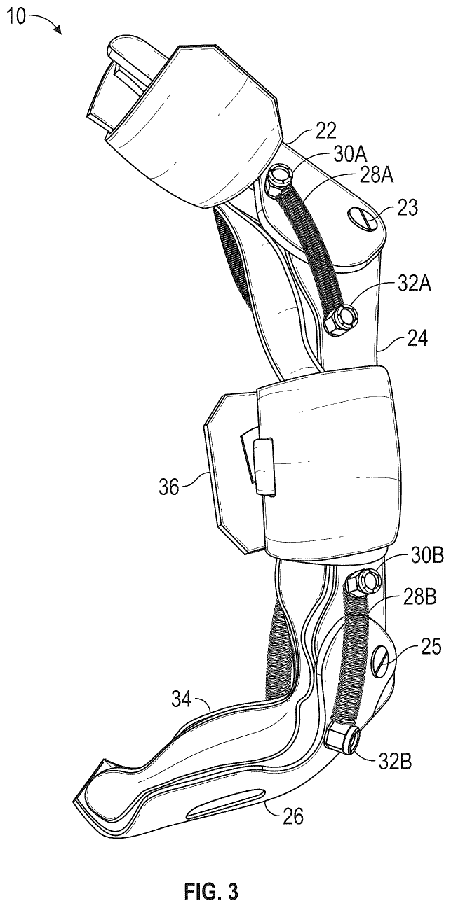

FIG. 3 shows additional aspects of at least some embodiments of a device 20 for use with four-legged animals, such as dogs. The device 20 includes padding 34 and straps 34 to better attach, hold, and provide comfort to the animal using the device. The padding 34 may be neoprene or other cushion material. The padding can be filled with a cushion like material, including air, fluids, solid, or semi-solid materials that will cushion and comfort the animal.

The straps are shown to be materials that include attachment portions. For example, the straps may be any material that can wrap, partially or wholly, around the device and the animal to hold the device in place. The attachments can be any manner, such as hook and loops, buttons, snaps, hooks, ties, or the like, which will hold the device in place for use with the animal.

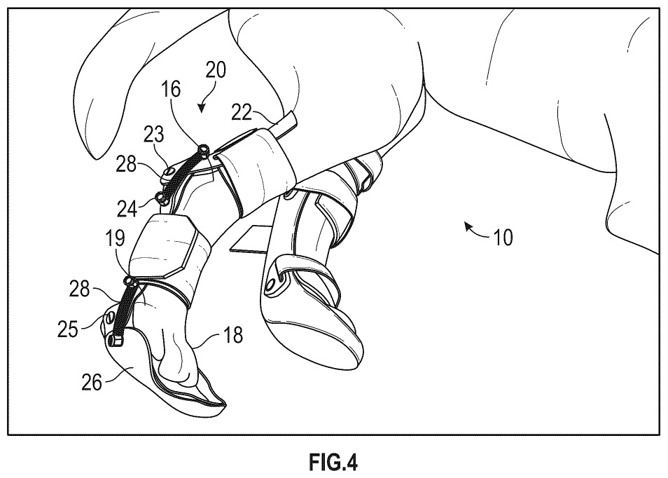

FIG. 4 shows devices on both pelvic limbs 11 of an animal, which is shown to be a dog. As can be seen in the figure, the device 20 has been custom fit for the animal, including the various anatomical portions of the patient. In addition, the straps 34 have been wrapped around the limb to hold the device 20 in place. While the patient in FIG. 4 includes devices on both rear limbs, it should be noted that this is not required. For example, a single device on a single limb could be used for the rehabilitation of the patient.

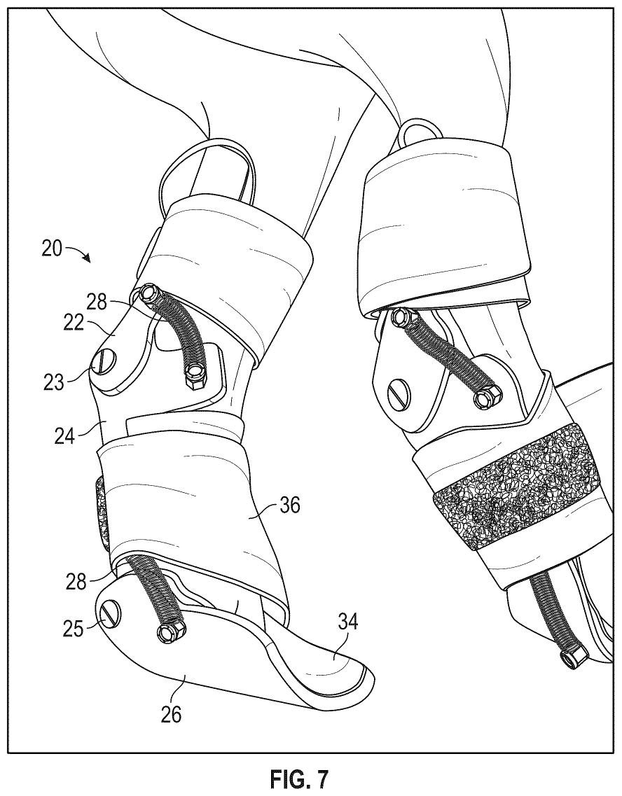

FIG. 7 is an enlarged view of FIG. 4 showing a close up of the animal 10 having multiple devices 20 on their pelvic limbs. As noted, the devices are strapped on and held in place via attachments to mitigate movement of the devices or the devices falling off, which could cause further injuries to the patient.

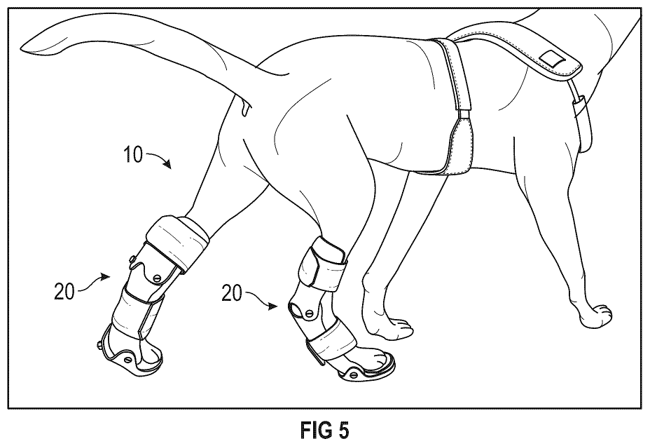

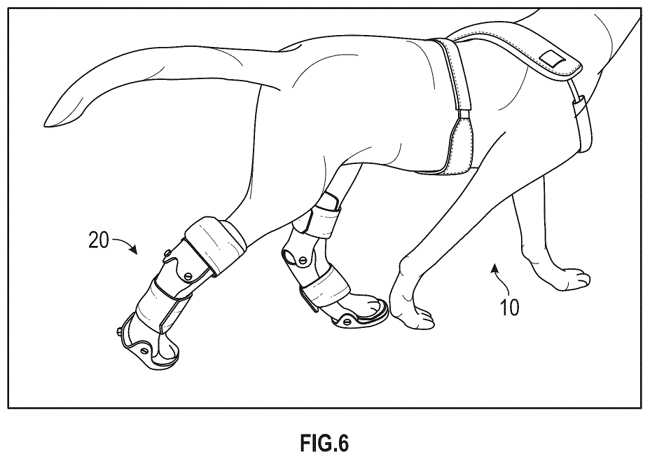

FIGS. 5 and 6 show the animal 10 using the devices 20. As noted in FIG. 1, certain conditions, such as musculoskeletal or neurological conditions, can affect a patient's ability to move in a normal manner. The devices 20 can aid in more natural movement. As shown in FIG. 1, the joints are not moving in a manner to bend the paw for placement on the ground. However, as shown in FIGS. 5 and 6, the device including the resilient springs 28 will urge the joints (both hock and metatarsal) to bend and flex in a manner that is similar to natural movement. The use of the tension springs allows flexing of the joints but will urge the joints in the non-flexed condition, which will mimic natural operation of the joints, which provides improvement for the movement of the patient. Put another way, the articulations of the rigid members are spring loaded to allow for natural motion of the hock and metatarsal/phalangeal joints in both flexion and extension.

Therefore, the movable splint device 20 can be produced and utilized in dogs with injury to the bone/muscle/tendon/ligament support structures as well as for nerve injury preventing unsupported standing and walking, such as is shown in FIGS. 5 and 6. This will mitigate the patient dragging their pelvic limbs (see, e.g., FIG. 1), which may be caused by injury and/or neurological condition.

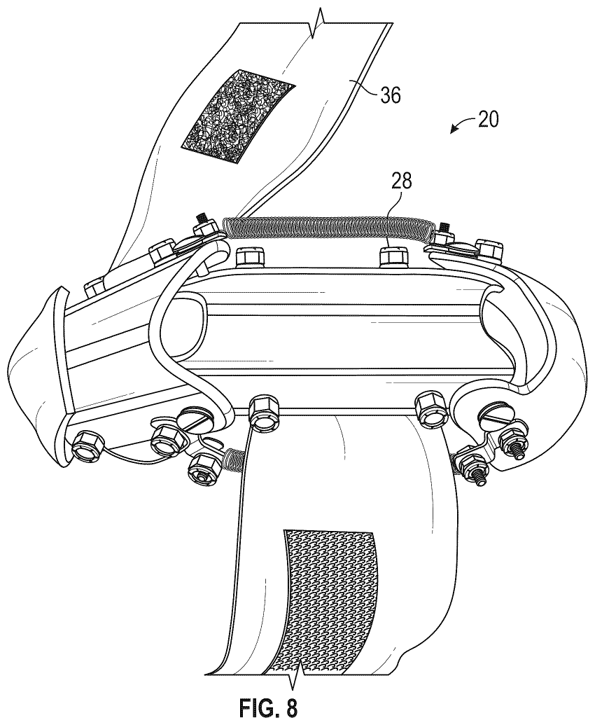

FIG. 8 is another view of another variation of a movable splint device 20, which features the improvements disclosed herein. For example, the device includes the multiple rigid members that are articulated and movable relative to one another via axes. In addition, resilient members, such as springs, are attached to the rigid portions to promote natural flexing and extension by the patient using the device.

It should be noted that the existing splints could also be manipulated or otherwise retrofitted with the resilient members to promote flexing and extension by a patient wearing the device to mimic a more natural movement of the patient wearing the device. The resilient springs urge the portions of the patient in the correct positions, which will encourage rehabilitation or even permanent usage of the device to aid in movement of the patient's limbs.

Therefore, a device 20 such as has been shown and/or described provides numerous improvements and advantages. The device 20 could be custom designed and fitted for individual patients. The advantages of the device include, but are not limited to, lightweight (devices can be less than ½ lb.), ease of placement, angled articulations which allow for support during the stance phase and more natural flexion motion during the flight phase of gait, and comfort. The device would be used during rehabilitation and retraining of the animal's movements. It should be appreciated that many variations, alternatives, and/or changes be made to any of the portions of any of the devices as shown and/or described. The device is not to be considered a single embodiment, and any of the aspects of any of the embodiments shown or described could be combined, even if not explicitly done herein, to create yet additional embodiments that provide the same or additional improvements and advantages for patients.

1. A splint that mimics natural dynamic joint movements for animals, comprising:
a first rigid splint member for supporting a first portion of an animal appendage;
a second rigid splint member rotatably connected to the first rigid splint member, the second rigid splint member configured to support a second portion of the animal appendage; and
a resilient member connecting the first and second rigid splint members, wherein the first and second rigid splint members able to rotate relative to one another upon application of a force and the resilient member urging the first and second rigid splint members to a non-rotated relationship upon easing of the force.

2. The movable splint of claim 1, further comprising a third rigid splint member rotatably connected to the second rigid splint member.

3. The movable splint of claim 2, further comprising a second resilient member connecting the second and third rigid splint members.

4. The movable splint of claim 3, wherein the third rigid splint member configured to receive a paw portion of the animal appendage.

5. The movable splint of claim 1, wherein the resilient member comprises a tension spring.

6. The movable splint of claim 1, wherein the resilient member comprises a plurality of resilient members each connecting the first and second rigid splint members.

7. The movable splint of claim 6, wherein the first and second rigid splint members supporting an ankle of an animal.

8. The movable splint of claim 1, further comprising padding on the first and second rigid splint members.

9. The movable splint of claim 8, wherein the padding comprises neoprene.

10. The movable splint of claim 1, further comprising one or more straps for holding the splint on the animal.

11. An animal splint, comprising:
a plurality of rigid splint members, each of the plurality of rigid splint members conforming to a portion of an animal's appendage;
wherein each of the plurality of rigid splint members is rotatably connected to an adjacent rigid splint member via corresponding axes of the rigid splint members; and
at least one resilient member between at least one pair of adjacent rigid splint members, the at least one resilient member urging the at least one pair of adjacent rigid splint members into an initial position.

12. The animal splint of claim 11, wherein the plurality of rigid splint members comprises an upper, a mid-level, and a lower rigid splint member, wherein the mid-level rigid splint member is rotatably connected to the upper rigid splint member at an upper position, and is rotatably connected to the lower rigid splint member at a lower position.

13. The animal splint of claim 12, comprising a first resilient member between the upper and mid-level rigid splint members, and a second resilient member between the mid-level and lower rigid splint members.

14. The animal splint of claim 12, wherein the lower rigid splint member includes a substantially planar portion and partially upstanding sidewalls configured to position a paw of an animal.

15. The animal splint of claim 11, wherein the at least one resilient member comprises a tension spring.

16. The animal splint of claim 11, further comprising padding in one or more of the plurality of rigid splint members.

17. The animal splint of claim 11, further comprising one or more straps associated with one or more of the plurality of rigid splint members.

18. The animal splint of claim 11, wherein the at least one resilient member comprises a rigid plastic.

19. An articulating animal splint, comprising:
a plurality of rigid splint members, each of the plurality of rigid splint members conforming to a portion of an animal's appendage;
wherein each of the plurality of rigid splint members is rotatably connected to an adjacent rigid splint member via corresponding axes of the rigid splint members;
at least one resilient member between at least one pair of adjacent rigid splint members, the at least one resilient member urging the at least one pair of adjacent rigid splint members into an initial position;
wherein the plurality of rigid splint members comprises an upper, a mid-level, and a lower rigid splint member, wherein the mid-level rigid splint member is rotatably connected to the upper rigid splint member at an upper position, and is rotatably connected to the lower rigid splint member at a lower position; and
padding in one or more of the plurality of rigid splint members.

20. The articulating animal splint of claim 19, wherein the at least one resilient member comprises:
a. a tension spring;
b. spring steel;
c. rubber; or
d. other elastomers.
